(19) United States
(12) Patent Application Publication
(10) Pub. No.: US 2017/0224412 A1
(43) Pub. Date: Aug. 10, 2017

(54) ENDOSCOPIC ENERGY TREATMENT INSTRUMENT AND ENDOSCOPIC SYSTEM

(71) Applicant: OLYMPUS CORPORATION, Tokyo (JP)

(72) Inventor: Akira MATSUI, Tokyo (JP)

(73) Assignee: OLYMPUS CORPORATION, Tokyo (JP)

(21) Appl. No.: 15/496,079

(22) Filed: Apr. 25, 2017

Related U.S. Application Data
(63) Continuation of application No. PCT/JP2014/080357, filed on Nov. 17, 2014.

(51) Int. Cl. A61B 18/14; A61B 90/70; A61B 18/12

(52) U.S. Cl. CPC A61B 18/1482; A61B 18/12; A61B 90/70; A61B 2576/00; A61B 2018/00601

(57) ABSTRACT

This endoscopic energy treatment instrument includes: a treatment instrument insertion part having flexibility and having a power reception part that is capacitively coupled by facing to a power transmission electrode arranged in a treatment instrument channel provided in an endoscope; a treatment part that is arranged on a distal end of the treatment instrument insertion part, and that uses electric power supplied from the power reception part to perform energy treatment; and a cleaning part that is arranged on the treatment instrument insertion part between the power reception part and the treatment part so as to be able to come into contact with the treatment instrument channel, the cleaning part being configured to clean an inside of the treatment instrument channel in accordance with a movement of the treatment instrument insertion part.

This application is a continuation application based on PCT Patent Application No. PCT/JP2014/080357, filed Nov. 17, 2014, the contents of which is incorporated herein by reference.

### BACKGROUND OF THE INVENTION

Field of the Invention

The present invention relates to an endoscopic energy treatment instrument and an endoscopic system, and in particular to an endoscopic energy treatment instrument that wirelessly supplies electric power to an energy treatment instrument.

Background Art

As endoscopic treatment instruments being inserted into a body cavity via a treatment instrument channel of an endoscope and treat biological tissue, there are a type of treatment instruments that use electric power energy when operating. For example, U.S. Pat. No. 7,824,407 discloses, as an energy treatment instrument which is inserted into a body through a treatment instrument channel of a flexible endoscope, a high frequency incision forceps that applies high frequency electric current to a biological tissue to perform a treatment. In the high frequency incision forceps disclosed in U.S. Pat. No. 7,824,407, a cable conductively connected to a high frequency power supply is connected to a connection terminal provided on an operation part. When a high frequency incision instrument is inserted in the close vicinity of an affected portion of a biological tissue, a return electrode is installed on a body surface so as to face to the high frequency incision instrument, and the high frequency power supply, the high frequency incision instrument, the affected portion, and the return electrode form a closed loop path for high frequency electric current.

In this manner, the conventional energy treatment instrument requires electric power to be supplied to the treatment instrument, and as a result, the energy treatment instrument and a power supply device are connected in a wired manner via a cable. However, if the energy treatment instrument and the power supply device are connected in a wired manner, the presence of the cable causes cumbersome handling of the operation of the energy treatment instrument.

As a method for solving this problem, U.S. Pat. No. 6,187,002 and U.S. Pat. No. 6,206,875 disclose devices that perform wireless electric power supply to an energy treatment instrument. U.S. Pat. No. 6,187,002 and U.S. Pat. No. 6,206,875 disclose devices that wirelessly supply electric power from a power transmission electrode provided on a trocar, to a power reception electrode of a capacitive cordless surgical instrument inserted into the trocar, via capacitive coupling.

### SUMMARY OF THE INVENTION

An endoscopic energy treatment instrument according to the present invention includes: a treatment instrument insertion part having flexibility and having a power reception part that is capacitively coupled by facing to a power transmission electrode arranged in a treatment instrument channel provided in an endoscope; a treatment part that is arranged on a distal end of the treatment instrument insertion part, and that uses electric power supplied from the power reception part to perform energy treatment; and a cleaning part that is arranged on the treatment instrument insertion part between the power reception part and the treatment part so as to be able to come into contact with the treatment instrument channel, the cleaning part being configured to clean an inside of the treatment instrument channel in accordance with a movement of the treatment instrument insertion part.

An endoscopic system according to the present invention includes: an endoscopic energy treatment instrument according to the present invention; and an endoscope having a treatment instrument channel having flexibility, and a power transmission part that is arranged along an outer circumferential surface of the treatment instrument channel, and that generates an alternating electric field to be applied to an interior of the treatment instrument channel.

### BRIEF DESCRIPTION OF DRAWINGS

FIG. 1 is a diagram showing an overall configuration of an endoscopic system provided with an endoscopic energy treatment instrument according to a first embodiment of the present invention.

FIG. 2 is a cross-sectional view showing a treatment instrument channel portion of an endoscope insertion part in the endoscopic system according to the first embodiment of the present invention.

FIG. 3 is a diagram showing an overall configuration of an endoscopic energy treatment instrument according to the first embodiment of the present invention.

FIG. 4 is a cross-sectional view showing a state of the endoscopic energy treatment instrument according to the first embodiment of the present invention being inserted into the endoscope insertion part.

FIG. 5 is an equivalent circuit diagram of the endoscopic system according to the first embodiment of the present invention.

FIG. 6 is a schematic diagram showing a use mode of the endoscopic energy treatment instrument according to the first embodiment of the present invention.

FIG. 7 is a schematic diagram showing a use mode of the endoscopic energy treatment instrument according to the first embodiment of the present invention.

FIG. 8 is a diagram showing a modified example of a cleaning part of the first embodiment, and is a cross-sectional view orthogonal to an axis line of the treatment instrument insertion part.

FIG. 9 is a diagram showing a modified example of the cleaning part of the first embodiment, and is a diagram seen from a direction orthogonal to a direction of the axis line of the treatment instrument insertion part.

FIG. 10 is a diagram showing a modified example of the cleaning part of the first embodiment, and is a diagram seen from a direction orthogonal to the direction of the axis line of the treatment instrument insertion part.

FIG. 11 is a diagram showing a modified example of the cleaning part of the first embodiment, and is a diagram seen from a direction orthogonal to the direction of the axis line of the treatment instrument insertion part.

FIG. 12 is a diagram showing, in a partial sectional view, a part of an endoscopic energy treatment instrument according to a second embodiment of the present invention.

FIG. 13 is a schematic diagram showing a use mode of the endoscopic energy treatment instrument according to the second embodiment of the present invention.

FIG. 14 is a partial sectional view showing a modified example of the endoscopic energy treatment instrument according to the second embodiment of the present invention.

FIG. 15 is a partial sectional view showing an endoscopic energy treatment instrument according to a third embodiment of the present invention.

FIG. 16 is a partial sectional view showing a state of an endoscopic energy treatment instrument according to a fourth embodiment of the present invention being inserted into a treatment instrument channel.

FIG. 17 is a diagram showing an overall configuration of an endoscopic system provided with the endoscopic energy treatment instrument according to the fourth embodiment of the present invention.

FIG. 18 is an equivalent circuit diagram of the endoscopic system according to the fourth embodiment of the present invention.

FIG. 19 is a partial sectional view of a distal end part of an endoscopic system of a modified example of the present invention.

### DETAILED DESCRIPTION OF THE INVENTION

### First Embodiment

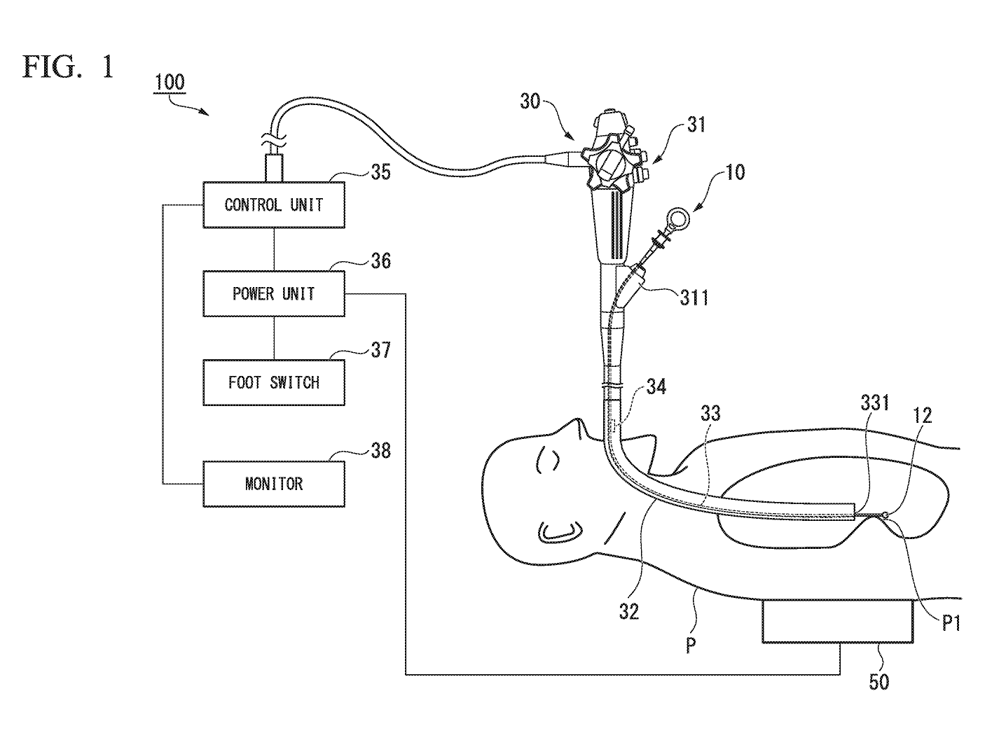

Hereunder, a first embodiment of the present invention is described, with reference to FIG. 1 through FIG. 5.

FIG. 1 is a diagram showing an overall configuration of an endoscopic system 100 provided with an endoscopic energy treatment instrument 10 according to the present embodiment (hereunder, referred to as “energy treatment instrument”).

As shown in FIG. 1, the endoscopic system 100 comprises a flexible endoscopic apparatus (hereunder, referred to as “endoscopic apparatus”) 30, an energy treatment instrument 10, and a return electrode 50. The endoscopic system 100 wirelessly performs electric power supply to the energy treatment instrument 10 without physical contact (connection) by means of a conductor through a capacitive coupling between a power transmission part (described later) provided in the endoscopic apparatus 30 and a power reception part (described later) provided in the energy treatment instrument 10.

The endoscopic apparatus 30 comprises an endoscope operation part 31, an endoscope insertion part 32, a power transmission part 34, a control unit 35, a power unit 36, a foot switch 37, and a monitor 38. In the present specification, a description is provided where, in the endoscopic apparatus 30, the side on which the endoscope operation part 31 is provided (the operator's hand side) is referred to as the base end side, and the side on which the endoscope insertion part 32 is provided is referred to as the distal end side.

The endoscope operation part 31 is provided with various switches and knobs for an operator to perform operations of the endoscope and operations of the endoscope insertion part.

The endoscope insertion part 32 is flexible and is formed in an elongated shape, and the base end part thereof is connected to the endoscope operation part 31. A commonly known endoscopic observation device that acquires a video image of the interior of a body, and an illuminating device are provided at the distal end of the endoscope insertion part 32.

A treatment instrument channel 33 is provided at the endoscope insertion part 32. The treatment instrument channel 33 is configured with a resin tube and is flexible, and allows the energy treatment instrument 10 or another commonly known treatment instrument to be inserted therethrough. The treatment instrument channel 33 is formed along the endoscope insertion part 32, and one end of the treatment instrument channel opens to the distal end plane of the endoscope insertion part 32 (a distal end opening 331) and the other end of the treatment instrument channel opens to a side part 311 of the endoscope operation part 31.

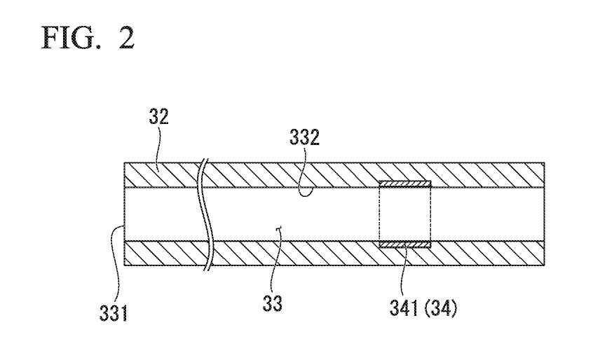

FIG. 2 is a cross-sectional view showing the treatment instrument channel 33 portion of the endoscope insertion part 32. As shown in FIG. 2, the power transmission part 34 is provided along the treatment instrument channel 33 in the endoscope insertion part 32. The power transmission part 34 is provided with a power transmission electrode 341 that generates an alternating electric field appropriate for high frequency electric power being supplied from the power unit 36, and an inductor (described later) that is connected in series to the power transmission electrode 341.

The power transmission electrode 341 is an electrode member that has an approximately 15 cm long cylindrical shape for example. The electrode member is provided so as to cover the treatment instrument channel 33 in a circumferential direction. The power transmission electrode 341 is provided at least in a part of a zone from the opening in the side part 311 of the endoscope operation part 31 of the treatment instrument channel, to the opening in the distal end plane of the endoscope insertion part 32 (distal end opening 331). As a material of the power transmission electrode 341, a conductor such as copper may be used for example. In a case where the power transmission electrode 341 is provided in the endoscope insertion part 32 having flexibility, the power transmission electrode also needs to be flexible. A flexible power transmission electrode 341 can be fabricated by forming a film of a metallic material such as copper on the outer circumferential surface of the treatment instrument channel 33, which is of a flexible tube, by means of a vapor deposition method or of a plating method for example.

The power transmission electrode 341 is covered with an insulating resin and is not exposed to an inner circumferential surface 332 of the treatment instrument channel 33, and an insulating material such as a resin is present between the power transmission electrode 341 and the inner circumferential surface 332 of the treatment instrument channel 33.

As shown in FIG. 1, the control unit 35 is connected to the endoscope operation part 31. The control unit 35 transmits signals that control operations of respective parts of the endoscopic apparatus 30, to perform control of the entire endoscopic apparatus 30.

The power unit 36 is connected to the control unit 35. In the endoscopic system 100, through the control unit 35, the power unit 36 supplies electric power required for driving the endoscopic apparatus 30, and electric power required for driving the energy treatment instrument 10.

The foot switch 37 is connected to the power unit 36, and the foot switch is provided to perform ON/OFF operations of energy output of high frequency electric power in the power unit 36. When an operator steps on the foot switch 37, electricity is conducted to the energy treatment instrument 10.

Although a foot switch is employed in the present embodiment, any form of switch that is capable of performing ON/OFF operations for conducting electricity to the energy treatment instrument may be employed.

The monitor 38 displays an image acquired by the endoscopic observation device (not shown in the figure) that is provided at the distal end of the endoscope insertion part 32.

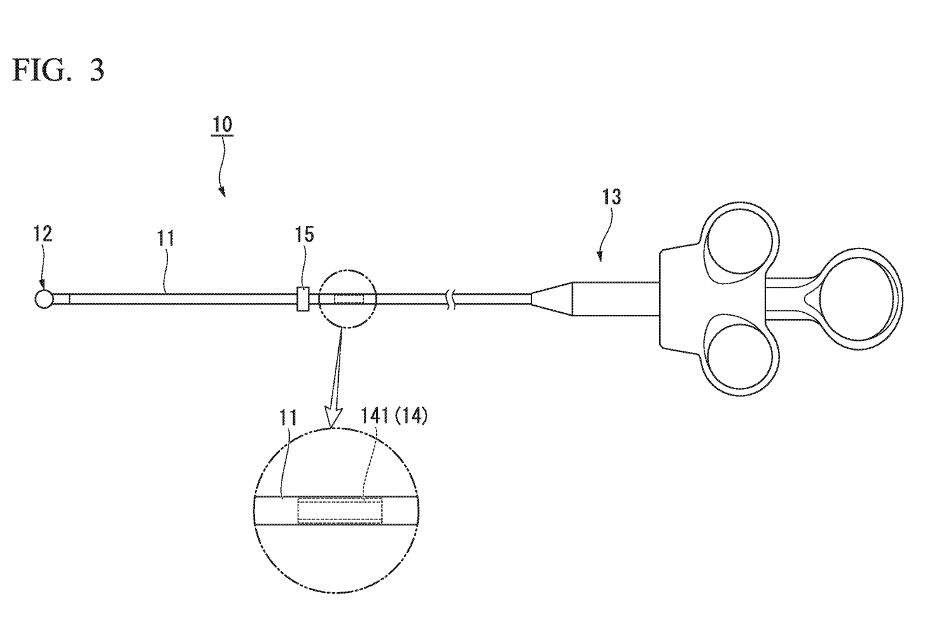

FIG. 3 is a diagram showing an overall configuration of the endoscopic energy treatment instrument 10. As shown in FIG. 1, the energy treatment instrument 10 is used for performing a treatment on a biological tissue (treatment target portion P1) of a patient P. The energy treatment instrument 10 is provided with a treatment instrument insertion part 11, a treatment part 12, a treatment instrument operation part 13, a power reception part 14, and a cleaning part 15.

The treatment instrument insertion part 11 has a level of flexibility so as to be able to be inserted through the treatment instrument channel 33, and the treatment instrument insertion part is formed in an elongated shape.

The treatment part 12 is provided at the distal end part of the treatment instrument insertion part 11. As a treatment part 12, a commonly known configuration for performing a treatment on a biological tissue (treatment target portion P1) with use of electric power energy, such as a high frequency knife, a forceps, and a needle, may be appropriately selected and employed. In the present embodiment, the treatment part 12 is a high frequency knife.

The treatment instrument operation part 13 is connected to the base end part of the treatment instrument insertion part 11. The treatment instrument insertion part 11 and the treatment part 12 is capable of advancing and retracting with respect to the treatment instrument channel 33 by operating the treatment instrument operation part 13.

The power reception part 14 is provided in the treatment instrument insertion part 11. The power reception part has a power reception electrode 141 and is connected to the treatment part 12 by means of wiring not shown in the figure. The power reception electrode 141 is a cylindrical electrode provided along an outer circumferential surface of the treatment instrument insertion part 11. The power reception electrode, for example, is formed with use of a conductive body such as copper. The power reception electrode 141 may be fabricated as with the power transmission electrode 341, for example, by forming a metal film on the outer circumferential surface of the treatment instrument insertion part 11. The power reception electrode 141, which is formed with a metal film, has flexibility. The outer surface of the power reception electrode 141 is covered with an insulating material such as a resin, in order to ensure electrical insulation properties.

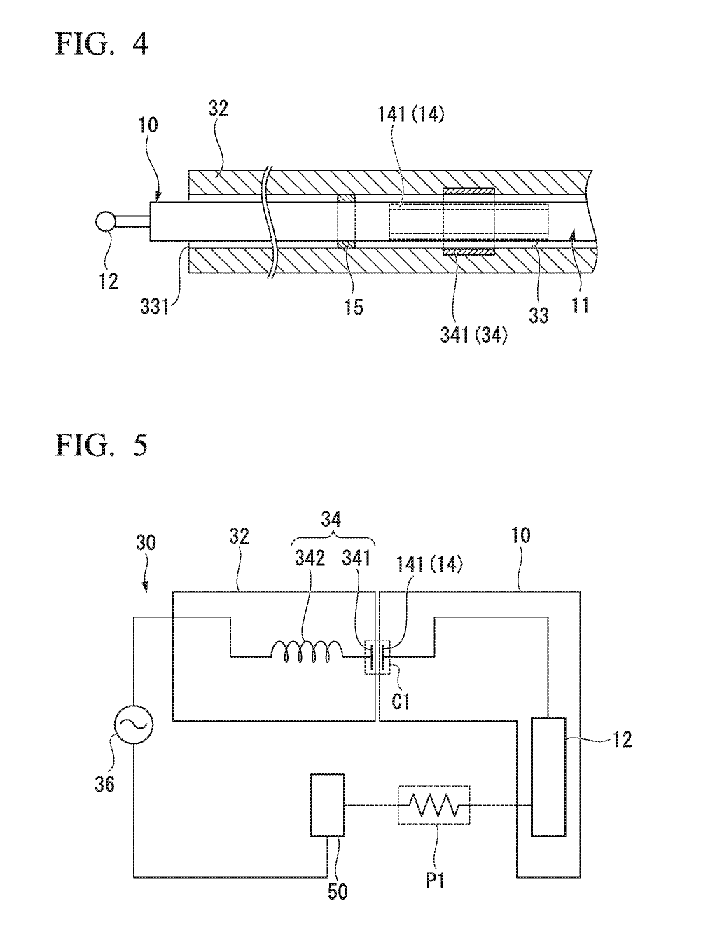

FIG. 4 is a cross-sectional view showing a state of the energy treatment instrument 10 being inserted into the endoscope insertion part 32. As shown in FIG. 4, when the treatment instrument insertion part 11 is inserted into the treatment instrument channel 33 to a position where the treatment part 12 projects from the distal end opening 331 of the treatment instrument channel 33, the power reception electrode 141 and the power transmission electrode 341 which is provided in the treatment instrument channel 33, substantially face to each other.

The power reception electrode 141 only needs to be present at a position where at least a part thereof can face to the power transmission electrode 341 when the treatment part 12 projects from the distal end opening 331 of the treatment instrument channel 33. Therefore, the power reception electrode 141 only needs to be provided in at least part of the treatment instrument insertion part 11 in their length direction. However, the treatment part 12 in some cases advances and retracts by several centimeters within the treatment instrument channel 33 when treatment is being performed. Therefore, it is preferable that the power transmission electrode and the power reception electrode are configured so that the entire lengthwise range of the power transmission electrode 341 always faces to the power reception electrode 141 even if the treatment part 12 is advanced or retracted in the treatment instrument channel 33 when treatment is being performed.

Accordingly, an axial direction length of the power reception electrode 141 is preferably greater by several centimeters than an axial direction length of the power transmission electrode 341.

As shown in FIG. 3 and FIG. 4, the cleaning part 15 is provided on an outer circumferential part of the treatment instrument insertion part 11. This cleaning part 15 in the present embodiment is provided on the more distal side than the power reception part 14.

In the present embodiment, the cleaning part 15 is provided so as to project radially outward from the treatment instrument insertion part 11 around a circumferential direction of the treatment instrument insertion part 11. A protrusion length of the cleaning part 15 from the outer circumferential surface of the treatment instrument insertion part 11 is set so that at least an outer circumferential edge part of the cleaning part 15 comes in contact with the inner circumferential surface of the treatment instrument channel 33 when the cleaning part 15 is placed at the position facing to the power transmission part 34 as a result of an advancing or retracting movement of the treatment instrument insertion part 11. That is to say, the cleaning part 15 is configured with an elastic member that has an outer diameter equal to or larger than an inner diameter of the treatment instrument channel 33, and that is able to, at least, deform into a shape along the inner diameter of the treatment instrument channel 33.

The cleaning part 15 may be formed integrally with the outer circumferential part of the treatment instrument insertion part 11, or may be formed by attaching another member on an outer circumference of the treatment instrument insertion part 11. The cleaning part 15 of the present embodiment is formed integrally with the outer circumferential part of the treatment instrument insertion part 11, by processing a part of the resin, such as polytetrafluoroethylene (PTFE), which forms the treatment instrument insertion part 11, into a protruding shape. In a case of attaching another member on the outer circumference of the treatment instrument insertion part 11 to form the cleaning part, another ring-shaped member of a soft material such as soft rubber and urethane sponge may be attached on the outer circumferential surface of the treatment instrument insertion part 11 by means of an adhesive material.

As shown in FIG. 1, the return electrode 50 is connected to the power unit 36, and is arranged so as to come in contact with the body surface of the patient P by being attached on the body surface of the patient P or by being installed on a bed. That is to say, the energy treatment instrument 10 of the present embodiment is a monopolar (monopolar type) energy treatment instrument that conducts electricity with the return electrode 50 via the patient P when the treatment part 12 comes in contact with the treatment target portion P1 of the patient P, to perform treatment.

Operations when in use of the endoscopic system 100 according to the present embodiment configured in the manner described above are described.

First, the operator inserts the energy treatment instrument 10 from the opening of the side part 311 of the endoscope operation part 31. The operator causes the treatment instrument insertion part 11 to advance through the treatment instrument channel 33 until the treatment part 12 projects from the distal end opening 331 of the treatment instrument channel 33. At this time, the cleaning part 15 first approaches the power transmission part 34, which is provided within the treatment instrument channel 33. As the treatment instrument insertion part 11 advances, the cleaning part 15 slides on and passes through the region on the inner circumferential surface 332 of the treatment instrument channel 33 where the power transmission electrode 341 is arranged. As a result, even in a case where a foreign substance such as dust is adhered on the inner circumferential surface 332 of the treatment instrument channel 33 in the close vicinity of the power transmission electrode 341, the foreign substance is pushed out to the distal end side by the cleaning part 15.

When the treatment part 12 projects from the distal end opening 331, then as shown in FIG. 4, the power reception part 14 and the power transmission part 34 face to each other across the insulating material therebetween. At this time, the cleaning part 15 ensures that there is no foreign substance between the power reception part 14 and the power transmission part 34.

FIG. 5 is an equivalent circuit diagram of the endoscopic system 100 in the state where the power reception part 14 and the power transmission part 34 face to each other. Since the power transmission electrode 341 and the power reception electrode 141 are arranged to face to each other in the state where the power reception part 14 and the power transmission part 34 face each other, a capacitor C1 is formed by means of capacitive coupling between these two electrodes. The power transmission part 34 is configured with the power transmission electrode 341 and an inductor 342 connected in series to the power transmission electrode 341. The inductor 342 and the capacitor C1 constitute an LC resonant circuit. When the operator operates the foot switch 37 (FIG. 1) in the state where the treatment part 12 is brought into contact with the treatment target portion P1, the power unit 36 supplies high frequency electric power to the power transmission part 34. The high frequency electric power flows to the treatment part 12 via the capacitor C1, and it travels through the treatment target portion P1 and flows to the return electrode 50. As a result, it is possible to perform treatment on the treatment target portion P1 with use of the treatment part 12. That is to say, electric power supply to the energy treatment instrument 10 is wirelessly performed without physical contact (connection) by means of a conductor between the power transmission part 34 and the power reception part 14, making it possible to perform treatment.

Frequency of the high frequency electric power supplied from the power unit 36 can be set in a range from approximately 100 kHz to approximately 100 MHz. It is preferable that the frequency of the high frequency electric power is selected from frequencies that are approved under regulations, and it is, for example, 13.56 MHz. By configuring the resonance frequency of the LC resonant circuit constituted by the capacitor C1 and the inductor 342 of the power transmission part 34 to match with the frequency of the high frequency electric power supplied from the power unit 36, electric power is efficiently supplied from the power transmission part to the power reception part. The efficiency of electric power input from the power supply part to the circuit shown in FIG. 5 may be increased by providing an impedance matching circuit in either the endoscopic apparatus 30 or the power unit 36, and matching the output impedance of the power supply part with the input impedance of the circuit shown in FIG. 5.

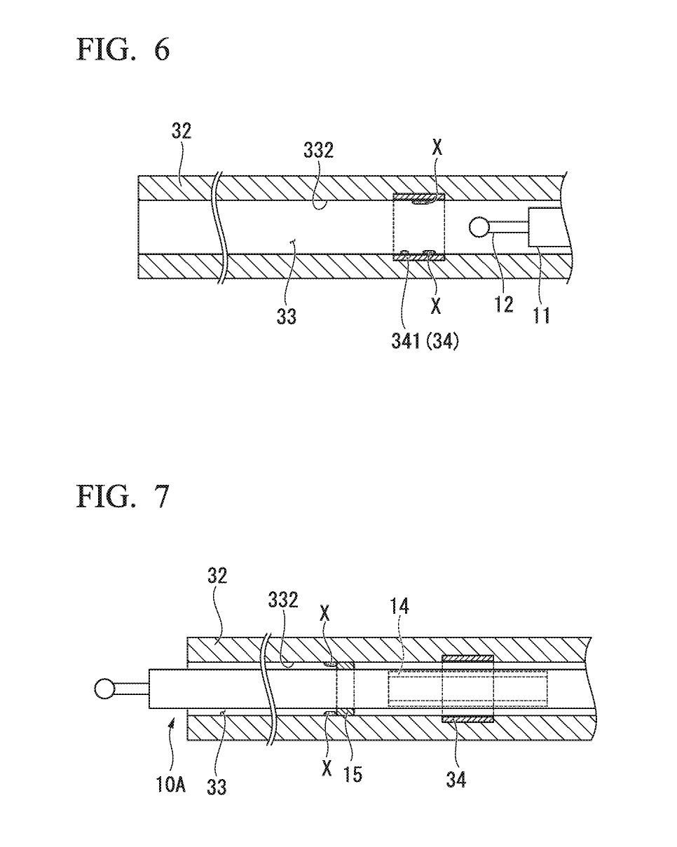

When treating the treatment target portion P1, the energy treatment instrument 10 is swapped in order to sequentially use appropriate treatment instruments according to the content of the treatment. When removing the energy treatment instrument 10 from the treatment instrument channel 33 for swapping, body fluid and tissue or the like adhered on the treatment part 12 and the treatment instrument channel 11 may become adhered as a foreign substance on the inner surface of the treatment instrument channel 33 in some cases. As shown in FIG. 6, if a foreign substance X becomes adhered within the range on the inner circumferential surface 332 of the treatment instrument channel 33 where the power transmission part 34 is provided, the capacitance of the capacitor C1, which is formed when the power transmission part 34 and the power reception part 14 face to each other, varies in accordance with the adhesion state of the foreign substance (the area of adhesion, the quantity of adhesion, and the type of adhesion). However, in the endoscopic system 100 of the present embodiment, a cleaning part is provided also on the energy treatment instrument to be newly inserted (hereunder, referred to as “swap energy treatment instrument”). Therefore, when the swap energy treatment instrument is inserted into the treatment instrument channel 33, then as with a movement similar to that described above, the cleaning part 15 provided on the swap energy treatment instrument 10A pushes out and removes the foreign substance X from the inner circumferential surface 332 in the range where the power transmission part 34 is provided, as shown in FIG. 7. Then, the power reception part 14 of the swap energy treatment instrument 10A and the power transmission part 34 face to each other, thereby forming a capacitor C1.

In this manner, in the endoscopic system 100, even if a foreign substance X becomes adhered on the inner circumferential surface 332 of the treatment instrument channel 33 when removing the energy treatment instrument 10 in use, the adhered foreign substance X is removed at least from the range where the power transmission part 34 is provided, by the cleaning part 15 of the swap energy treatment instrument 10A to be inserted next. Therefore, even in the case where energy treatment instruments are swapped frequently when performing treatment, it is ensured that no foreign substance is present between the power transmission part 34 and the power reception part 14, and it is possible to suppress changes in impedance of the circuit including the power transmission part 34 and power reception part 14 with respect to the power unit 36. As a result, variation in electric power transmission to the energy treatment instrument 10 that occur as impedance variation is capable of being suppressed, and it is possible to perform stable wireless power feeding from the endoscopic apparatus 30 to the energy treatment instrument 10.

As described above, according to the endoscopic system 100 of the present embodiment which is provided with the energy treatment instrument 10, since the cleaning part 15 is provided, stable wireless power feeding is always possible.

Since the endoscopic system of the present embodiment is not necessary to connect electrical wiring such as a cable to the energy treatment instrument 10 for power feeding, the operability of the energy treatment instrument is improved.

Since the cleaning part 15 is arranged closer to the distal end than the power reception part 14, when the energy treatment instrument 10 is inserted into the treatment instrument channel 33, the cleaning part 15 always stays in contact with and slides on the inner circumferential surface 332 of the treatment instrument channel 33 in the close vicinity of the power transmission part 34, before the power reception part 14 faces to the power transmission part 34. Therefore, any foreign substance between the power transmission part 34 and the power reception part 14 can be reliably removed, before supplying high frequency electric power from the power unit 36 to the power transmission part when preforming a treatment. That is to say, any foreign substance in the close vicinity of the power transmission electrode 341 within the treatment instrument channel 33 can be automatically removed by the cleaning part 15 by just inserting the energy treatment instrument 10, and the operator is not required to perform any special operation to remove the foreign substance. As a result, it is possible to improve the usability of the endoscopic system 100.

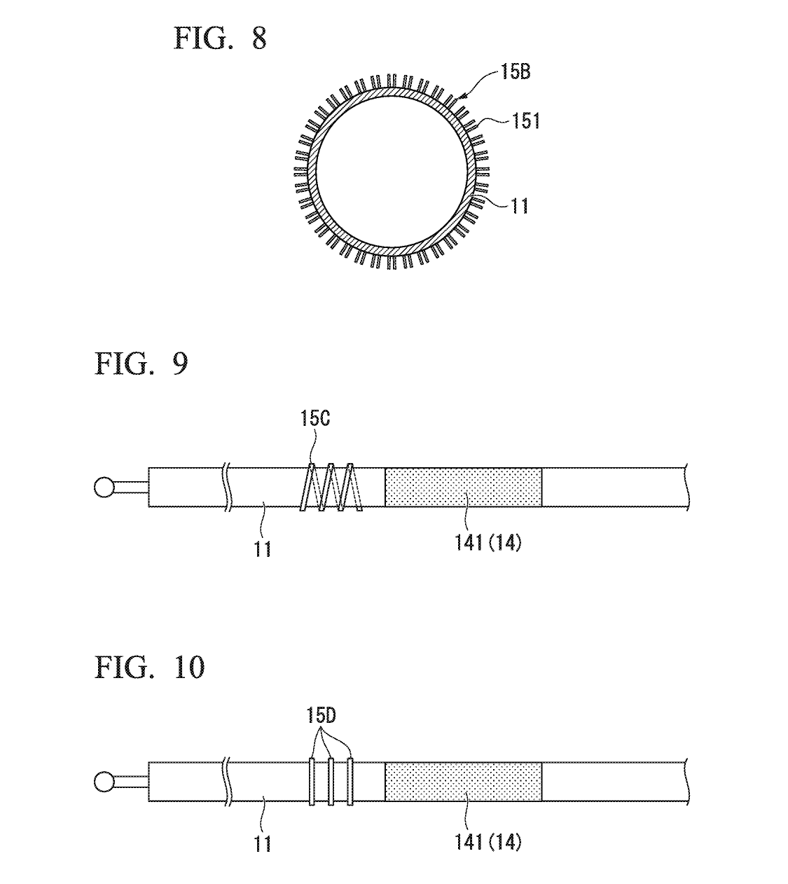

In the present embodiment, the cleaning part 15 is not limited to the configuration of being provided on the entire outer circumferential surface of the treatment instrument insertion part 11. FIG. 8 to FIG. 11 show modified examples of the energy treatment instrument, in which configurations of the cleaning part differ. FIG. 8 is diagram showing modified example of the cleaning part of the first embodiment, and is cross-sectional view orthogonal to the axis line of the treatment instrument insertion part 11.

In the modified example shown in FIG. 8, there is provided a brush type cleaning part 15B in which a plurality of long and thin resin projections 151 that project radially outward from the outer circumferential surface of the treatment instrument insertion part 11 are arranged around the circumferential direction of the treatment instrument insertion part 11.

In addition, although omitted in the figure, the cleaning part may be employed with no particular limitation only if a configuration that can come in contact with and slide on the inner circumferential surface 332 of the treatment instrument channel 33, such as a cleaning part formed with a thin plate-shaped valve disc that extends around the circumferential direction and toward the radially outside of the treatment instrument insertion part 11.

In the modified example shown in FIG. 9, a band-shaped protrusion part that constitutes a cleaning part 15C is provided so as to be helically wound on the outer circumferential surface of the treatment instrument insertion part 11.

In the modified example shown in FIG. 10, three ring-shaped cleaning parts 15D are provided at intervals in a longitudinal axis direction of the treatment instrument insertion part 11.

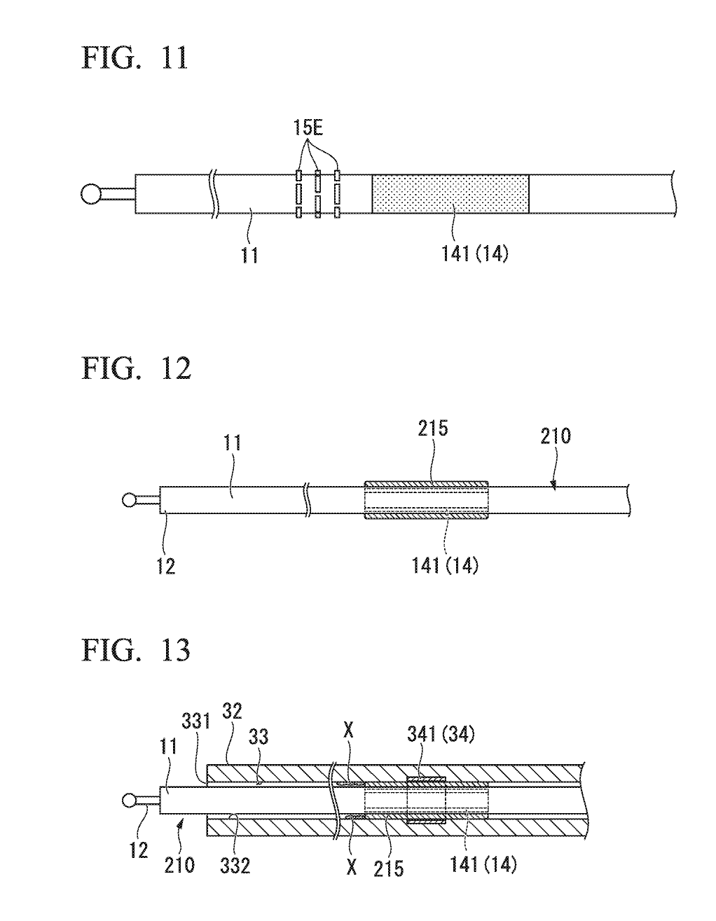

Cleaning parts 15E of the modified example shown in FIG. 11 each have a plurality of protrusion parts provided at intervals in the circumferential direction of the treatment instrument insertion part 11, and as with the modified example shown in FIG. 10, these are provided in three sets at intervals in the longitudinal axis direction of the treatment instrument insertion part 11. In the example shown in FIG. 11, by shifting the interval phase of the protrusion part in the circumferential direction of the treatment instrument insertion part 11 in each set of the longitudinal axis direction, the cleaning parts 15E comes in contact with the entire inner circumferential surface of the treatment instrument channel as a whole. In the modified examples shown in FIG. 9, FIG. 10, and FIG. 11, since the length of the cleaning part in the longitudinal axis direction of the treatment instrument insertion part 11 is increased, the level of foreign substance removal is higher at the time of insertion. Furthermore, since the cleaning parts on the outer circumferential surface of the treatment instrument insertion part 11 are at constant intervals or are arranged being divided into several pieces, the flexibility of the treatment instrument insertion part is not impaired even if the length of the cleaning parts is increased. In addition, the configurations of the modified examples above may be appropriately combined to configure the cleaning part.

In the present embodiment, the configuration in which the power transmission electrode 341 and the power reception electrode 141 are covered with an insulating resin has been shown as an example. However, capacitive coupling is still possible provided that insulation is maintained between the power transmission electrode 341 and the power reception electrode 141 when the power transmission electrode 341 and the power reception electrode 141 are positioned to face to each other. Therefore, only the surface of at least either one of the power transmission electrode and the power reception electrode needs to be covered with a material of insulation properties.

Although the inductor 342 is arranged in the power transmission part 34 in the present embodiment, the arrangement location is not limited to this location. The inductor may be arranged so as to connect to the power reception electrode 141 of the power reception part 14 and may be form an LC resonant circuit that includes the capacitor C1. Moreover, also in a case where no inductor 342 is present, that is to say, where no LC resonant circuit that includes the capacitor C1 is formed, an action and an effect similar to those of the above examples can still be demonstrated.

### Second Embodiment

A second embodiment of the present invention is described with reference to FIG. 12 and FIG. 13. In the embodiment described below, the constituents similar to those of the energy treatment instrument of the first embodiment above in terms of function and structure, are given the same reference symbols as those of the above embodiment, and descriptions that overlap with the above embodiment are omitted.

FIG. 12 is a diagram showing, in a partial sectional view, a part of an energy treatment instrument 210 according to the present embodiment. FIG. 13 is a schematic diagram showing a use mode of the energy treatment instrument 210. The energy treatment instrument 210 according to the present embodiment differs from the first embodiment in the configuration of a cleaning part 215. That is to say, as shown in FIG. 12, the cleaning part 215 is provided on the entire area of the surface where the power reception electrode 141 is provided in the outer circumferential surface of the treatment instrument insertion part 11. The cleaning part covers the entire power reception electrode 141. The cleaning part 215 is configured in a manner such that a soft resin-made cylindrical member having an area substantially equal to that of the power reception electrode 141 is attached on the treatment instrument insertion part 11 so as to cover the power reception electrode 141.

According to the energy treatment instrument 210 of the present embodiment, an effect similar to that of the first embodiment can be achieved. Furthermore, since the cleaning part 215 is provided so as to cover the surface of the power reception electrode 141, the cleaning part 215 is present between the power reception electrode 141 and the power transmission electrode 341 and there is no gap therein when the power reception electrode 141 is arranged in a position facing to the power transmission electrode 341, as shown in FIG. 13. As a result, a foreign substance is suppressed to adhere between the power reception electrode 141 and the power transmission electrode 341 after a foreign substance X has been removed from the inner circumferential surface 332 of the treatment instrument channel 33.

In the present embodiment, the cleaning part may be formed integrally with the treatment instrument insertion part 11 by forming a part, which covers the power reception electrode 141, of the resin configuring the treatment instrument insertion part 11 thick so as to project radially outward.

Moreover, if the cleaning part is configured using a high-dielectric material, the capacitance of the capacitor C1, which is formed by the power transmission electrode 341 and the power reception electrode 141, is capable of increasing. If the capacitance of the capacitor C1 increases, influence of changes in parasitic capacitance, which may occur on the wiring path including the treatment part 12 in some cases when performing treatment, becomes more unlikely, and more stable electric power transmission becomes possible. Moreover, since the voltage occurring at the capacitor C1 can be lowered, insulation properties of the apparatus is easily to ensure. Examples of high-dielectric materials include a fluorine resin such as polytetrafluoroethylene (PTFE), which has a relative permittivity ∈r=2.1, a silicone rubber, which has a relative permittivity ∈r=3 to 4, and a fluorocarbon rubber, which has a relative permittivity ∈r=6 to 7.

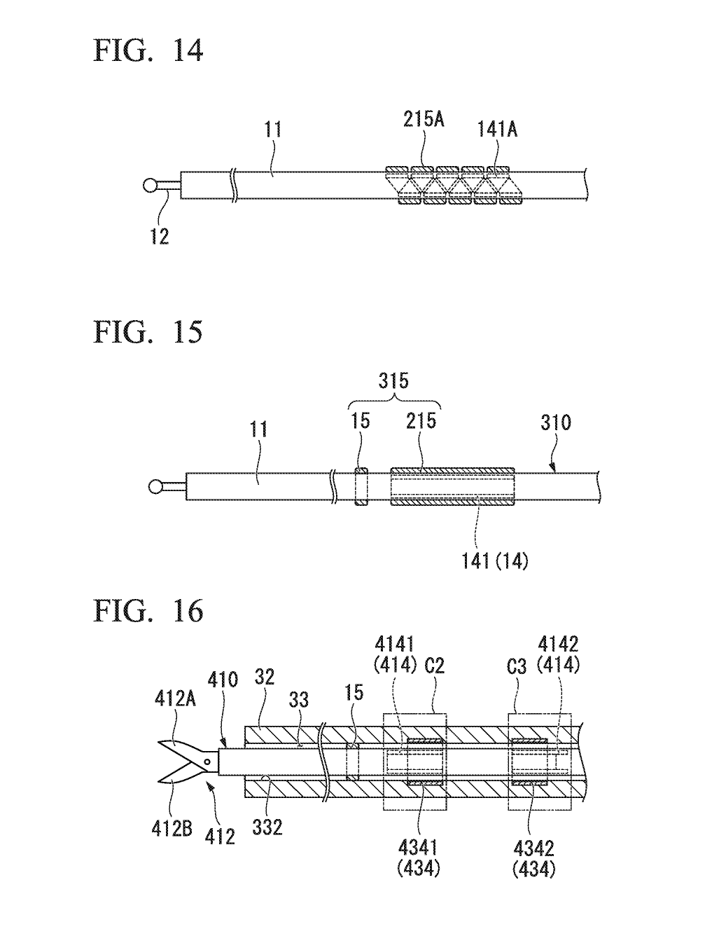

A cross-sectional view of a modified example of the present embodiment is shown in FIG. 14. In the example shown in FIG. 14, a power reception electrode 141A is of a spiral type shape. That is to say, the power reception electrode 141A is formed in a manner such that a thin plate/band-shaped conductor is helically wound at a size substantially equal to the outer circumferential diameter of the treatment instrument insertion part 11. The cleaning part 215A is provided in a manner such that a thin plate/band-shaped insulating resin in a shape similar to that of the power reception electrode 141A is helically wound so as to cover the entire power reception electrode 141A.

According to the present modified example, an effect similar to that of the second embodiment can be achieved. Furthermore, if the power reception electrode 141A and the cleaning part 215A are configured in a spiral shape as practiced in the present modified example, the power reception electrode 141A and the cleaning part 215A are easily made to track the bend of the treatment instrument insertion part 11. As a result, it is possible to suitably perform operations without impairing the flexibility of the treatment instrument insertion part 11 and the endoscope insertion part 32 with the treatment instrument insertion part 11 inserted therein. Furthermore, by employing a power reception electrode 141A and a cleaning part 215A such as ones in the present modified example, if by any chance a foreign substance is present, the foreign substance gets taken into the gap in the cleaning part 215A, i.e., a portion of the inner circumferential surface 332 of the treatment instrument channel 33 where the power reception electrode 141A is not provided. Therefore it is possible to prevent the foreign substance from becoming adhered between the power reception electrode 141A and the power transmission electrode 341.

The cleaning part 215 of the present embodiment does not always need to cover the entire surface of the power reception electrode 141A, and only needs to be of a shape that covers at least the outer circumferential part including the distal end part of the power reception electrode 141A.

### Third Embodiment

A third embodiment is described, with reference to FIG. 15. FIG. 15 is a partial sectional view showing an energy treatment instrument 310 according to the present embodiment. A cleaning part 315 of the energy treatment instrument 310 has the cleaning part 15 of the first embodiment and the cleaning part 215 of the second embodiment in the axial direction of the treatment instrument insertion part 11. In this manner, by having the cleaning parts 15 and 215 arranged at two locations, namely on the outer circumferential part and on the distal end side of the power reception electrode 141, it is possible to more reliably remove a foreign substance X, and wireless power feeding performance can be stabilized further.

### Fourth Embodiment

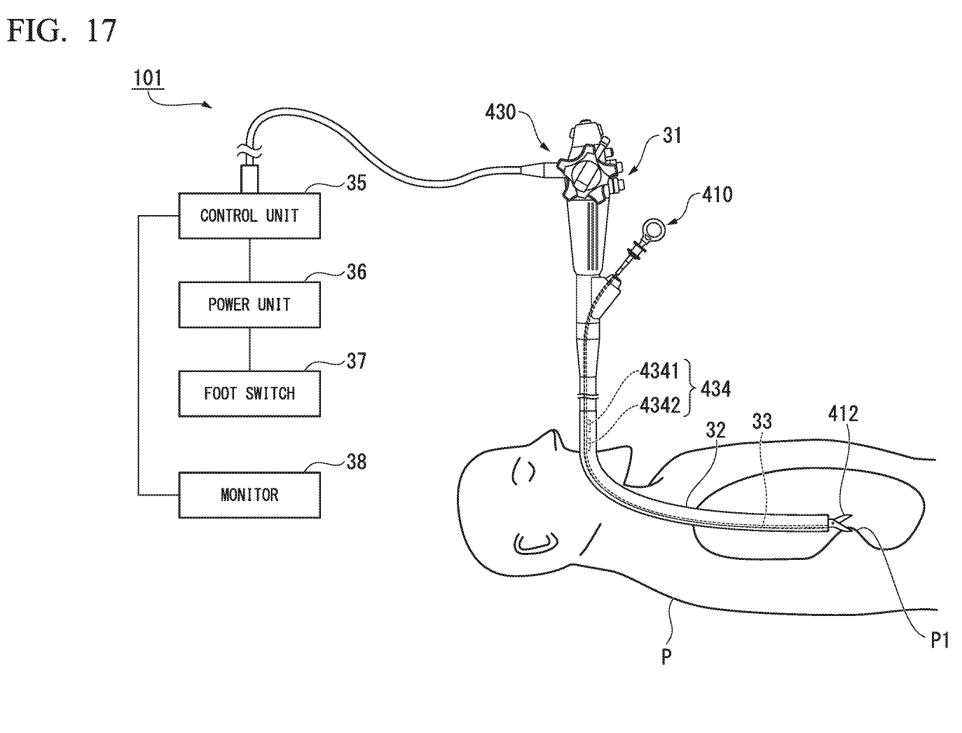

The respective embodiments described above are all for an endoscopic system provided with a monopolar treatment instrument. However, the present embodiment is an endoscopic system provided with a bipolar treatment instrument. An energy treatment instrument 410 according to the fourth embodiment of the present invention is described, with reference to FIG. 16 and FIG. 17. FIG. 16 is a partial sectional view showing a state of the energy treatment instrument 410 being inserted into a treatment instrument channel 33. FIG. 17 is a diagram showing an overall configuration of an endoscopic system 101 provided with the endoscopic energy treatment instrument 410. As shown in FIG. 17, the endoscopic system 101 comprises an endoscopic apparatus 430, and an energy treatment instrument 410.

The energy treatment instrument 410 is a bipolar (bipolar type) treatment instrument. As shown in FIG. 16, as a treatment part 412, the energy treatment instrument 410 is provided with a forceps having a pair of forceps pieces 412A and 412B.

As shown in FIG. 17, the endoscopic apparatus 430 has, on a power transmission part 434, two power transmission electrodes, namely a first power transmission electrode 4341 and a second power transmission electrode 4342. As shown in FIG. 16, the power reception part 414 of the energy treatment instrument 410 is provided with two power reception electrodes, namely a first power reception electrode 4141 and a second power reception electrode 4142.

As shown in FIG. 16, when the treatment instrument insertion part 11 is inserted into the treatment instrument channel 33 to a position where the treatment part 412 projects from the distal end opening 331 of the treatment instrument channel 33, the first power reception electrode 4141 and the first power transmission electrode 4341 substantially face to each other, and the second power reception electrode 4142 and the second power transmission electrode 4342 substantially face to each other.

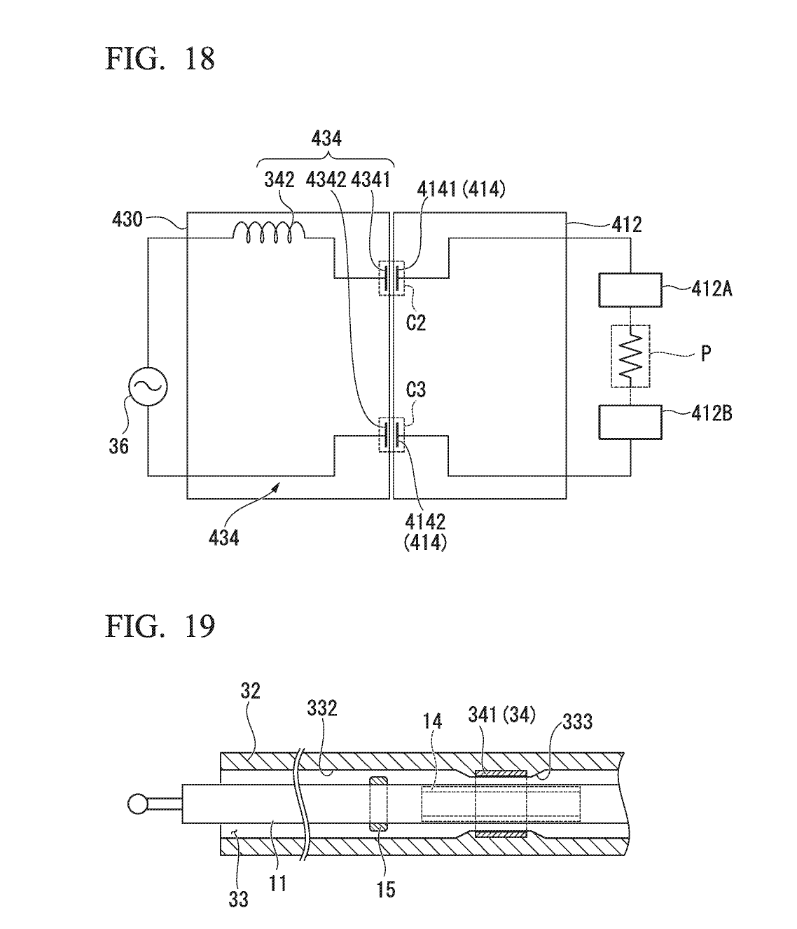

FIG. 18 is an equivalent circuit diagram of the endoscopic system 101 in the state where the power reception part 414 and the power transmission part 434 are faced to each other. The first power reception electrode 4141 is connected by means of wiring to one of the forceps pieces (first forceps piece 412A) of the treatment part 412. Similarly, the second power reception electrode 4142 is also connected to the other forceps piece (second forceps piece 412B) of the treatment part 412. In the endoscopic system 101, the first power transmission electrode 4341 and the first power reception electrode 4141 constitute a first capacitor C2, and the second power transmission electrode 4342 and the second power reception electrode 4142 constitute a second capacitor C3. When the operator steps on the foot switch 37 to perform electric power supply from the power unit 36, high frequency electric current flows through the first capacitor C2, the first forceps piece 412A, the second forceps piece 412B, and the second capacitor C3, and treatment can be performed on a tissue sandwiched between the first forceps piece 412A and the second forceps pieces 412B. The cleaning part 15 of the present embodiment is provided on the outer circumferential part of the insertion part of the energy treatment instrument 410. This cleaning part 15 is not limited to the shape nor arrangement shown in FIG. 16, and a configuration similar to that of each of the other embodiments described above, may be applied thereto.

Also in the endoscopic system 101 of the present embodiment, stable power feeding can be realized as with the respective embodiments described above. The present invention may be also suitably applied to an endoscopic system that is provided with a bipolar treatment instrument.

While the embodiments of the present invention have been described, a specific constitution of the invention is not limited to the above embodiments, and combination of the constituents in each embodiment may be changed, and removal or various modifications may be made to each constituent, without departing from the scope of the invention.

For example, as shown in FIG. 19, the channel of a portion 333 of the treatment instrument channel 33 where the power transmission electrode 341 is arranged may be formed with a reduced diameter. With this type of configuration, the cleaning part 15 can be brought into contact with the treatment instrument channel 33 only in the portion 333 where the power transmission part 34 is arranged, and the slide resistance in portions other than the portion 333 can be made low. As a result, friction that occurs as a result of movements in the insertion direction (insertion and/or removal) and rotating operations performed in the treatment instrument channel 33 of the treatment instrument insertion part 11 is reduced, and the operability of the energy treatment instrument 510 at the time of performing treatment is improved. Moreover, the operator can recognize that the cleaning part 15 has reached the power transmission electrode 341, by the tactile feel transmitted to the treatment instrument operation part 13.

The present invention is not limited to the above description, and is limited only by the claims appended hereto.

What is claimed is:

1. An endoscopic energy treatment instrument comprising:
a treatment instrument insertion part having flexibility and having a power reception part that is capacitively coupled by being opposed to a power transmission electrode arranged in a treatment instrument channel provided in an endoscope;
a treatment part that is arranged on a distal end side of the treatment instrument insertion part, and that uses electric power supplied from the power reception part to perform energy treatment; and
a cleaning part that is arranged on the treatment instrument insertion part between the power reception part and the treatment part so as to be able to come into contact with the treatment instrument channel, the cleaning part being configured to clean an inside of the treatment instrument channel in accordance with a movement of the treatment instrument insertion part.

2. The endoscopic energy treatment instrument according to claim 1, wherein the cleaning part is helically wound in a plurality of time with a distance in a longitudinal axis direction on the treatment instrument insertion part.

3. The endoscopic energy treatment instrument according to claim 1, wherein a plurality of the cleaning part is arranged.

4. The endoscopic energy treatment instrument according to claim 1, wherein the cleaning part is arranged so as to cover at least a distal end part of the power reception part.

5. The endoscopic energy treatment instrument according to claim 4, wherein the cleaning part has a shape similar to that of the power reception part, and is arranged at a position that covers the power reception part as a whole.

6. The endoscopic energy treatment instrument according to claim 1, wherein the cleaning part is configured with a high-dielectric material.

7. The endoscopic energy treatment instrument according to claim 1, wherein the cleaning part is arranged on the power reception part and on more distal side of treatment instrument insertion part than the power reception part.

8. An endoscopic system comprising:
an endoscopic energy treatment instrument according to claim 1; and
an endoscope including
a treatment instrument channel having flexibility, and
a power transmission part that is arranged along an outer circumferential surface of the treatment instrument channel, and that generates an alternating electric field to be applied to an interior of the treatment instrument channel.
